(19) United States
(12) Patent Application Publication
(10) Pub. No.: US 2023/0196249 A1
(43) Pub. Date: Jun. 22, 2023

(54) SYSTEM FOR CALCULATING COST OF LABOR PRODUCTIVITY LOSS, METHOD FOR CALCULATING COST OF LABOR PRODUCTIVITY LOSS, AND PROGRAM FOR CALCULATING COST OF LABOR PRODUCTIVITY LOSS

(71) Applicant: KINJIRO CO., LTD., Tokyo (JP)

(72) Inventors: Minoru KAMURA, Tokyo (JP); Kenshi KAMURA, Tokyo (JP); Tatsuya KUNII, Tokyo (JP); Takashi NAKAMURA, Tokyo (JP)

(73) Assignee: KINJIRO CO., LTD., Tokyo (JP)

(21) Appl. No.: 17/922,137

(22) PCT Filed: Apr. 23, 2021
(86) PCT No.: PCT/JP2021/016495

§ 371 Date: Oct. 28, 2022

(30) Foreign Application Priority Data
Apr. 30, 2020 (JP) 2020-080639

(51) Int. Cl. G06Q 10/0639; G16H 10/60

(52) U.S. Cl. CPC G06Q 10/0639; G16H 10/60

(57) ABSTRACT

This system for calculating the cost of labor productivity loss comprises: an employment information storage unit which stores data pertaining to employment information for each employee; a medical examination result storage unit which stores data pertaining to a medical examination for each employee; a stress check result storage unit which stores data pertaining to a stress check for each employee; a setting data storage unit which stores setting data; a cost of labor productivity loss calculation unit which calculates the cost of labor productivity loss from item data stored in the employment information storage unit, item data stored in the medical examination result storage unit, and item data stored in the stress check result storage unit; and an output unit which outputs the calculated cost of labor productivity loss.

### CROSS-REFERENCE TO RELATED APPLICATION(S)

This application is a U.S. National Stage filing under 35 U.S.C. § 371 of International Patent Application No. PCT/JP2021/016495, filed on Apr. 23, 2021, which in turn claims priority under PCT Article 8 and/or 35 U.S.C. § 119(a) to Japanese Patent Application No. 2020-080639, filed on Apr. 30, 2020. The entire content of Japanese Patent Application No. 2020-080639 is incorporated by reference herein.

### TECHNICAL FIELD

The present disclosure relates to a system for calculating cost of labor productivity loss, a method for calculating cost of labor productivity loss, and a program for calculating cost of labor productivity loss.

### BACKGROUND

In recent years, the Ministry of Economy, Trade and Industry has implemented the “Certified Health & Productivity Management Outstanding Organizations Recognition Program” with the aim of promoting management initiatives that address employee health management as a management issue. In order to maintain health and sound management of an organization, it is required that the amount of health-related costs be known. Health-related costs include medical costs, an injury and sickness benefit and a workers’ compensation benefit for disabilities, an absenteeism loss cost, and a presenteeism loss cost (e.g., see Non-Patent Literature 1).

A known system collects item data on attendance, item data on results of medical examinations, and item data on results of stress checks (e.g., see Patent Literature 1). A management server forming the system manages the item data on attendance, item data on results of medical examinations, and item data on results of stress checks. The item data on the stress checks generally follows the questionnaire “Brief Job Stress Questionnaire (57 items)” recommended by the Ministry of Health, Labour and Welfare (e.g., see Non-Patent Literature 2). As a method for calculating a stress value in accordance with the “Brief Job Stress Questionnaire (57 items)”, a simple total score (e.g., see Non-Patent Literature 3) and a standardized score using a raw score conversion table are known. There is also a known method of weighting and scoring each item data on stress checks so that an employee with low motivation and an employee with poor mental health are more consistent with the tendency of stress check scores of the employees (e.g., see Patent Literature 2).

Here, among the health-related costs, the injury and sickness benefit, the medical cost, and the worker’s compensation benefit are clear from data held by a health insurance association or a company, but the absenteeism loss cost and the presenteeism loss cost need to be calculated through one method or another. Non-Patent Literature 4 exemplifies calculation using “Absenteeism loss cost = Number of sick days (absence from work) per year × Wage (yen)” and “Presenteeism loss cost = presenteeism loss ratio × Wage (yen)”. The presenteeism loss ratio is calculated using the World Health Organization Health and Work Performance Questionnaire (WHO-HPQ) scale”. In Non Patent Literature 5, the number of sick days (absence from work) of absenteeism is obtained through a questionnaire.

### CITATIONS LIST

### Patent Literature

Patent Literature 1: Japanese Laid-Open Patent Publication No. 2017-068831
Patent Literature 2: Japanese Laid-Open Patent Publication No. 2017-102673

### Non-Patent Literature

Non Patent Literature 1: “Healthy Workforce 2010 and Beyond, 2009”, [online], Nov. 30, 2009, Partnership for Prevention and U.S. Chamber of Commerce, [searched on Nov. 26, 2019], Internet <URL: https://www.uschamber.com/sites/default/files/documents/files/HealthyWorkforce2010FINA LElectronicVersion111709.pdf>
Non Patent Literature 2: “Brief Job Stress Questionnaire (57 items)”, [online], the Ministry of Health, Labour and Welfare, [searched on Nov. 26, 2019], Internet <URL: https://www.mhlw.go.jp/bunya/roudoukijun/anzeneisei12/dl/stresscheck_j.pdf>
Non Patent Literature 3: “Stress Check System Implementation Manual Based on Industrial Safety and Health Act”, [online], revised in July 2019, the Ministry of Health, Labour and Welfare, [searched on Nov. 26, 2019], Internet <URL: https://www.mhlw.go.jp/content/000533925.pdf>
Non Patent Literature 4: “Visualization of Health Issues through Collaboration between Insurers and Business Owners Based on the Framework of ‘Health Management’”, [online], Mar. 31, 2015, TOKYO MARINE NICHIDO HEALTH INSURANCE ASSOCIATION, [searched on Nov. 26, 2019], Internet <URL: https://www.mhlw.go.jp/file/06-Seisakujouhou-12400000-Hokenkyoku/houkoku12.pdf>
Non Patent Literature 5: “Visualization of Health Problems by Framework of Health Management”, [online], University Hospital Medical Information Network Center, [searched on Nov. 26, 2019], Internet <URL: http://square.umin.ac.jp/hpm/hpmmethod.html>

### SUMMARY

### Technical Problem

In management where employee health management is a management issue, it is important to grasp health-related costs for early detection of factors such as a decline in motivation and mental health disorders and implementing appropriate measures. However, in a conventional method or system, a questionnaire method targeting employees is used as a method to quantify an absenteeism loss cost and a presenteeism loss cost, which form health-related costs. It is difficult to say that a loss cost calculated solely on the basis of an employee’s uncertain memory and an ambiguous evaluation dependent on individual determination criteria reflects the reality of productivity-related factors.

An object of the present disclosure is to provide a method for calculating cost of labor productivity loss, a program for calculating cost of labor productivity loss, and a system for calculating cost of labor productivity loss that improve accuracy in the calculation of the cost of labor productivity loss.

### Solutions to Problem

A system for calculating cost of labor productivity loss that solves the above problem includes an employment attendance information storage unit, a medial examination result storage, a stress check result storage unit, a setting data storage unit, a labor productivity loss cost calculation unit, and an output unit. The employment attendance information storage unit stores pieces of item data on employment attendance information for employees in association with employee codes respectively assigned to the employees. The medical examination result storage unit stores pieces of item data on a medical examination for each of the employees in association with the corresponding employee code. The stress check result storage unit stores pieces of item data on a stress check for each of the employees in association with the corresponding employee code. The setting data storage unit stores setting data set for calculating cost of labor productivity loss. The labor productivity loss cost calculation unit calculates cost of labor productivity loss from the item data stored in the employment attendance information storage unit, the item data stored in the medical examination result storage unit, and the item data stored in the stress check result storage unit. The output unit outputs the cost of labor productivity loss calculated by the labor productivity loss cost calculation unit to a screen or a file in a tabular format or a graphical format.

A method for calculating cost of labor productivity loss that solves the above problem includes calculating cost of labor productivity loss with a labor productivity loss cost calculation unit by using pieces of item data on employment attendance information for employees in association with employee codes respectively assigned to the employees, pieces of item data on a medical examination for each of the employees in association with the corresponding employee code, pieces of item data on a stress check for each of the employees in association with the corresponding employee code, and setting data set for calculating cost of labor productivity loss. The method further includes outputting the calculated cost of labor productivity loss with an output unit to a screen or a file in a tabular format or a graphical format.

A program for calculating cost of labor productivity loss that solves the above problem causes a computer to function as an employment attendance information storage unit, a medical examination result storage unit, a stress check result storage unit, a setting data storage unit, a labor productivity loss cost calculation unit, and an output unit. The employment attendance information storage unit stores pieces of item data on employment attendance information for employees in association with employee codes respectively assigned to the employees. The medical examination result storage unit stores pieces of item data on a medical examination for each of the employees in association with the corresponding employee code. The stress check result storage unit stores pieces of item data on a stress check for each of the employees in association with the corresponding employee code. The setting data storage unit stores setting data set for calculating cost of labor productivity loss. The labor productivity loss cost calculation unit calculates cost of labor productivity loss from the item data stored in the employment attendance information storage unit, the item data stored in the medical examination result storage unit, and the item data stored in the stress check result storage unit. The output unit outputs the cost of labor productivity loss output by the labor productivity loss cost calculation unit to a screen or a file in a tabular format or a graphical format.

With each of the above configurations, for example, the overtime loss cost can be calculated from the number of overtime hours of employees and the hourly unit price stored in the employment attendance information storage unit. Further, for example, the absenteeism loss cost can be calculated from the paid leave and absence from work due to sickness stored in the employment attendance information storage unit, and the average individual daily unit price included in the setting data. Moreover, for example, the presenteeism loss cost can be calculated from the medical examination result, the stress check result, and the average individual monthly unit price included in the setting data. Therefore, errors based on an employee’s uncertain memory and an ambiguous evaluation dependent on individual determination criteria are reduced. Further, the accuracy in the calculation of the cost of labor productivity loss is improved.

In the system for calculating the cost of labor productivity loss, the labor productivity loss cost calculation unit may include an absenteeism loss cost calculation unit that calculates an absenteeism loss cost on the basis of the item data stored in the employment attendance information storage unit, an overtime loss cost calculation unit that calculates an overtime loss cost on the basis of the item data stored in the employment attendance information storage unit, and a presenteeism loss cost calculation unit that calculates a presenteeism loss cost by using the item data stored in the medical examination result storage unit and the item data stored in the stress check result storage unit as samples for specifying a presenteeism loss rate. Further, the labor productivity loss cost calculation unit may calculate, as the cost of labor productivity loss, a sum of the absenteeism loss cost calculated by the absenteeism loss cost calculation unit, the presenteeism loss cost calculated by the presenteeism loss cost calculation unit, and the overtime loss cost calculated by the overtime loss cost calculation unit.

In the system for calculating the cost of labor productivity loss, the labor productivity loss cost calculation unit may include an absenteeism loss cost calculation unit that calculates an absenteeism loss cost on the basis of the item data stored in the employment attendance information storage unit, an overtime loss cost calculation unit that calculates an overtime loss cost on the basis of the item data stored in the employment attendance information storage unit, a lifestyle disease loss cost calculation unit that calculates a lifestyle disease loss cost on the basis of the item data stored in the medical examination result storage unit, a stress loss cost calculation unit that calculates a stress loss cost on the basis of the item data stored in the stress check result storage unit, and an overtime loss cost calculation unit that calculates an overtime loss cost on a basis of the item data stored in the employment attendance information storage unit. The labor productivity loss cost calculation unit may calculate the cost of labor productivity loss with following Equations 1 and 2.

$$\text{Presenteemism lost cost = Lifestyle disease loss cost + Stress loss cost}$$

$$\begin{matrix}\text{Cost of labor productivity loss = Absenteeism loss cost +} \\ \text{Presenteeism loss cost + Overtime loss cost}\end{matrix}$$

With such configurations, the cost of labor productivity loss can be calculated taking into consideration the decrease in labor productivity caused by the mental health disorders of the employees in addition to the decrease in labor productivity caused by the physical health disorders of the employees.

In the above system for calculating the cost of labor productivity loss, the stress loss cost calculation unit may calculate a stress value of each of employees on the basis of the item data stored in the stress check result storage unit. When an employee of which the stress value is larger than a stress upper limit boundary value included in the setting data is defined as a person with high stress, the stress loss cost calculation unit may calculate the stress loss cost with Equation 3 shown below on the basis of a number of people with high stress that is the number of persons with high stress, a stress loss rate stored in the setting data storage unit, an average individual monthly unit price that is an average amount of salary paid to one employee per month, and the number of labor productivity loss cost calculation months.

$$\begin{matrix}\text{Stress loss cost = Number of people with high stress} \times \\ \text{Number of calculation months} \times \\ \text{Average individual monthly unit price} \times \text{Stress loss rate}\end{matrix}$$

With this configuration, the stress loss cost can be calculated in correspondence with the magnitude of the total of the stress values of the employees.

In the above system for calculating the cost of labor productivity loss, when an employee determined to have a lifestyle disease on the basis of the item data stored in the medical examination result storage unit and a criteria determination table for each of medical examination items is defined as a person with a lifestyle disease, the lifestyle disease loss cost calculation unit may calculate the lifestyle disease loss cost with Equation 4 shown below on the basis of a number of people with a lifestyle disease that is the number of persons with a lifestyle disease, a lifestyle disease loss rate stored in the setting data storage unit, an average individual monthly unit price that is an average amount of salary paid to one employee per month, and the number of labor productivity loss cost calculation months.

$$\begin{matrix}\text{Lifestyle disease loss cost =} \\ \text{Number of people with a lifestyle disease} \times \text{Number of} \\ \text{calculations months} \times \text{Average individual montly unit price} \\ \times \text{Lifestyle disease loss rate}\end{matrix}$$

With this configuration, the lifestyle disease loss cost can be calculated in correspondence with the magnitude of the total of lifestyle disease values in the company.

In the system for calculating the cost of labor productivity loss, the absenteeism loss cost calculation unit may calculate the absenteeism loss cost with Equation 5 shown below on the basis of the number of paid leave days due to poor health of an employee and the number of absence days obtained from the employment attendance information storage unit, and an average individual daily unit price that is an average amount of salary paid to one employee per day, in which the average individual daily unit price is included in the setting data.

$$\begin{matrix}\text{Absenteeism loss cost =} \\ \left(\text{Number of absence days + Number of paid leave days due}\right) \\ \left(\text{to poor health}\right) \times \text{Average individual daily unit price}\end{matrix}$$

With this configuration, the cost of labor productivity loss can be calculated without performing an additional questionnaire to calculate the absenteeism loss cost.

In the system for calculating the cost of labor productivity loss, the overtime loss cost calculation unit may calculate the overtime loss cost with Equation 6 shown below on the basis of overtime hours including overtime work and legal overtime of an employee obtained from the employment attendance information storage unit:

$$\begin{matrix}\text{Overtime loss cost =} \\ \text{Overtime hours of all employees in calculation period} \times \\ \text{Average individual hourly unit price}\end{matrix}$$

### Advantageous Effects of the Embodiments

The present embodiments improve the accuracy in the calculation of the cost of labor productivity loss. For example, it is possible to calculate and present the cost of labor productivity loss for the entire company, each department, and each employee. This allows a manager, a person in charge of human resources and labor, a department head, an industrial physician, and a health management adviser to take measures to improve the working environment and implement improvement, as well as measures to improve employees’ lifestyle diseases and mental health disorders, from their respective perspectives. As a result, improvement in labor productivity can be expected, leading to improvement in corporate profit. In addition, it is possible to detect excessive overtime hours, an increase in the number of people in poor health, an increase in the number of people absent from work on the day, and the like. Further, measures can be taken to improve working motivation. In addition, increases and decreases in lifestyle diseases and mental health disorder can be measured. This improves the work productivity.

### BRIEF DESCRIPTION OF DRAWINGS

FIG. 1 is a block diagram illustrating a configuration of a first embodiment of a system for calculating cost of labor productivity loss.

FIG. 2 is a diagram illustrating the configuration of an employment attendance information record according to the embodiment.

FIG. 3 is a diagram illustrating the configuration of a medical examination result record according to the embodiment.

FIG. 4 is a diagram illustrating the configuration of a stress check result record according to the embodiment.

FIG. 5 is a flowchart illustrating a process for calculating a standard value in the embodiment.

FIG. 6 is a flowchart illustrating a procedure for calculating a medical examination numerical value in the embodiment.

FIG. 7 is an explanatory diagram illustrating a flow of reading and writing of data performed in criteria determination records in the embodiment.

FIG. 8 is a flowchart illustrating a process for calculating a stress loss cost in the embodiment.

FIG. 9 is a flowchart illustrating a process for calculating a lifestyle disease loss cost in the embodiment.

FIG. 10 is a flowchart illustrating a process for calculating an absenteeism loss cost according to the embodiment.

FIG. 11 is a block diagram illustrating a configuration of a system for calculating cost of labor productivity loss according to a second embodiment.

FIGS. 12A and 12B are schematic diagrams illustrating results of a questionnaire on work execution efficiency according to the embodiment.

FIGS. 13A to 13C are schematic diagrams illustrating examples of the questionnaire on work execution efficiency in the embodiment.

FIG. 14 is a flowchart illustrating a process for calculating a presenteeism loss rate according to the embodiment.

FIG. 15 is a flowchart illustrating a process for calculating a presenteeism loss cost according to a modification of the embodiment.

FIG. 16 is a block diagram illustrating a configuration of a system for calculating cost of labor productivity loss according to a fourth embodiment.

FIG. 17 is a schematic diagram illustrating a presenteeism loss rate definition table according to the embodiment.

FIG. 18A is a schematic diagram illustrating a determination result concerning presenteeism loss determination items in the embodiment. FIG. 18B is a schematic diagram illustrating a result of counting the number of people corresponding to the presenteeism loss determination items in the embodiment.

FIG. 19 is a flowchart illustrating a process for calculating a presenteeism loss cost in the embodiment.

### DESCRIPTION OF EMBODIMENTS

### First Embodiment

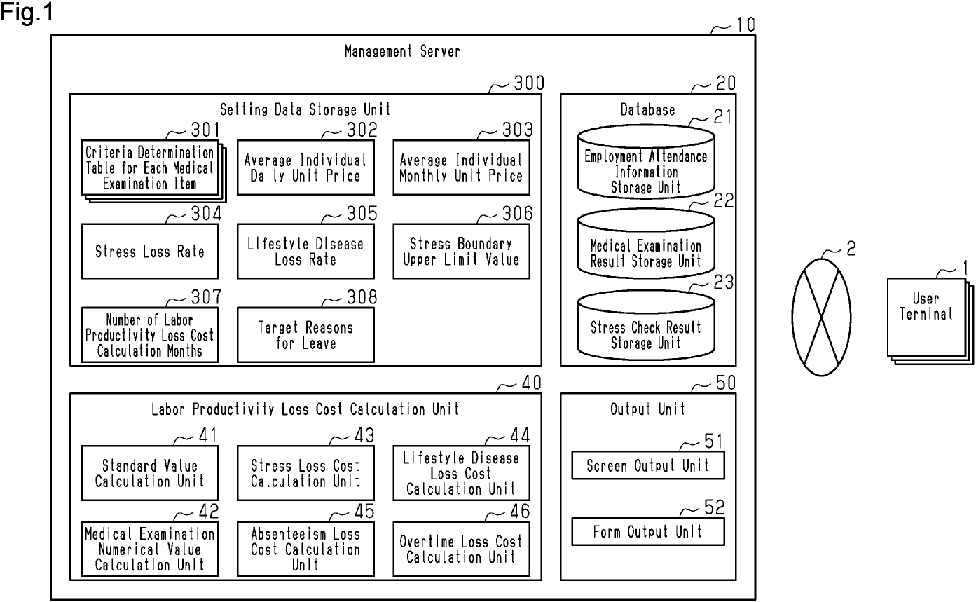

A first embodiment of a system for calculating cost of labor productivity loss, a program for calculating cost of labor productivity loss, and a method for calculating cost of labor productivity loss will be described with reference to FIGS. 1 to 10.

As illustrated in FIG. 1, the system for calculating the cost of labor productivity loss includes a management server 10. The management server 10 is connected to the plurality of user terminals 1 via a network 2. The management server 10 stores the program for calculating the cost of labor productivity loss and executes the program for calculating the cost of labor productivity loss to calculate the cost of labor productivity loss. The management server 10 can be configured as a circuitry including 1) one or more processors that execute various types of processing according to a computer program (software), 2) one or more dedicated hardware circuits such as an application-specific integrated circuit (ASIC) that execute at least some of the various types of processing, or 3) a combination thereof. The processor includes a central processing unit (CPU) and memories such as random-access memory (RAM) and read-only memory (ROM), and the memory stores a program code or a command configured to cause the CPU to execute processing. The memory or computer-readable medium includes any available medium accessible by a general-purpose or special-purpose computer.

The user terminal 1 is, for example, a computer terminal of a desktop type, a notebook type, or a tablet type used by a person in charge of labor management of a company and includes an output unit and an input unit. The output unit has the functionality for outputting various types of information and includes a display, a printer, and the like. The input unit has the functionality for inputting various types of information and includes a keyboard, a pointing device, a communication interface, and the like. The user terminal 1 is configured to be accessible to files and the like that are saved and set to be shared on the management server 10 and other servers except for the management server 10. The user terminal 1 is configured to be able to download and store files and the like that are saved and set to be shared on the management server 10 and other servers except for the management server 10.

The management server 10 is a computer system formed by hardware such as a CPU, a ROM, a RAM, and a hard disk. The management server 10 includes a database 20, a setting data storage unit 300, a labor productivity loss cost calculation unit 40, and a server-side output unit 50.

The database 20 includes an employment attendance information storage unit 21, a medical examination result storage unit 22, and a stress check result storage unit 23. The employment attendance information storage unit 21 stores employment information of each employee. The medical examination result storage unit 22 stores information on a medical examination result of each employee. The stress check result storage unit 23 stores information concerning a stress check result of each employee.

The setting data storage unit 300 stores setting data of various settings, such as a numerical value serving as a condition for calculating the cost of labor productivity loss and a numerical value used for calculation. The setting data is data preset by a user of the system for calculating the cost of labor productivity loss in order to calculate the cost of labor productivity loss. The setting data includes a criteria determination table 301 for each medical examination item, an average individual daily unit price 302, an average individual monthly unit price 303, a stress loss rate 304, a lifestyle disease loss rate 305, a stress boundary upper limit value 306, the number of labor productivity loss cost calculation months 307, and target reasons for leave 308.

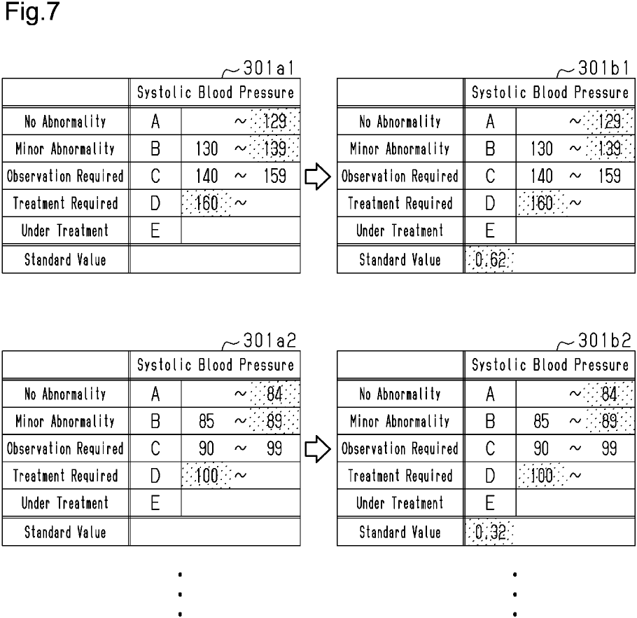

For example, as illustrated in a systolic blood pressure criteria determination table 301a1 in FIG. 7, each criteria determination table 301 stores a measured value range for determining the measured value at four levels of “A”, “B”, “C”, and “D” for the systolic blood pressure, which is the medical examination item, in association with each measured value.

The average individual daily unit price 302 is an average labor cost per day for all employees calculated on the basis of the financial statements. The average individual monthly unit price 303 is an average labor cost per month for all the employees calculated on the basis of the financial statements. The stress loss rate 304, the lifestyle disease loss rate 305, and the stress boundary upper limit value 306 are values that are the system eigenvalues or set by a user from the user terminal 1 and are arbitrary values that are considered appropriate on the basis of various research results. The number of labor productivity loss cost calculation months 307 is the number of months of a period subject to the calculation of the cost of labor productivity loss and is set by the user in accordance with the time when the stress check or the medical examination is performed, or is the system eigenvalue.

The target reasons for leave 308 are used to identify a leave to be used for calculating the absenteeism loss cost among leaves taken by the employee. The labor productivity loss cost calculation unit 40 uses the number of days in which the target reasons for leave 308 are used as reasons for leave among the number of days in which the employee has taken leaves to calculate an absenteeism loss cost. For example, in addition to expected leave such as maternity leave, childcare leave, leave due to hospitalization for medical treatment, condolence, and the like, the target reasons for leave 308 also include unexpected leaves such as a sudden sickness when catching a cold or poor health conditions due to a personal relationship at work, overwork, burnout syndrome, and the like.

The labor productivity loss cost calculation unit 40 includes a standard value calculation unit 41, a medical examination numerical value calculation unit 42, a stress loss cost calculation unit 43, a lifestyle disease loss cost calculation unit 44, an absenteeism loss cost calculation unit 45, and an overtime loss cost calculation unit 46. The standard value calculation unit 41 calculates a standard value for each medical examination item. The medical examination numerical value calculation unit 42 calculates a medical examination numerical value for each medical examination item. The stress loss cost calculation unit 43 calculates a stress loss cost that is included in a presenteeism loss cost. The lifestyle disease loss cost calculation unit 44 calculates a lifestyle disease loss cost that is included in the presenteeism loss cost. The absenteeism loss cost calculation unit 45 calculates an absenteeism loss cost.

Before the calculation of the cost of labor productivity loss, the standard value calculation unit 41 calculates the standard value of each medical examination item by using the criteria determination table 301 for each medical examination item. The standard value calculated by the standard value calculation unit 41 is stored into the criteria determination table 301 corresponding to the medical examination item of the standard value.

The medical examination numerical value calculation unit 42 calculates a medical examination numerical value of each medical examination item by using a medical examination result record 22a and the criteria determination table 301 for each medical examination item. The medical examination numerical value calculated by the medical examination numerical value calculation unit 42 is stored into the medical examination result storage unit 22. The medical examination numerical value calculated by the medical examination numerical value calculation unit 42 is used for calculating a lifestyle disease loss cost.

The stress loss cost calculation unit 43 calculates the stress loss cost by using a stress check result record 23a, the stress loss rate 304, and the average individual monthly unit price 303.

The lifestyle disease loss cost calculation unit 44 calculates the lifestyle disease loss cost by using the medical examination result record 22a, the lifestyle disease loss rate 305, and the average individual monthly unit price 303.

The absenteeism loss cost calculation unit 45 calculates the absenteeism loss cost by using an employment attendance information record 21a, the average individual daily unit price 302, and the target reasons for leave 308.

The overtime loss cost calculation unit 46 calculates the overtime loss cost by applying the employment attendance information record 21a and an average individual hourly unit price, which is derived from the average individual daily unit price 302, to Equation 6, which is shown below.

$$\begin{matrix}\text{Overtime loss cost =} \\ \text{Overtime hours of all employees in calculation period} \times \\ \text{Average individual hourly unit price}\end{matrix}$$

The server-side output unit 50 includes a screen output unit 51 and a form output unit 52. The screen output unit 51 outputs data including the absenteeism loss cost and the presenteeism loss cost calculated by the labor productivity loss cost calculation unit 40 to an external screen. The screen output unit 51 outputs the absenteeism loss cost and the presenteeism loss cost to the user terminal 1. The user terminal 1, to which the absenteeism loss cost and the presenteeism loss cost have been output, displays the costs on the display in a tabular format or a graphical format. The form output unit 52 outputs data including the absenteeism loss cost and the presenteeism loss cost calculated by the labor productivity loss cost calculation unit 40 to a form. The form output unit 52 prints the absenteeism loss cost and the presenteeism loss cost in a tabular format or a graphical format through a printer (not illustrated) via the network 2 to output the cost to the form.

### Database

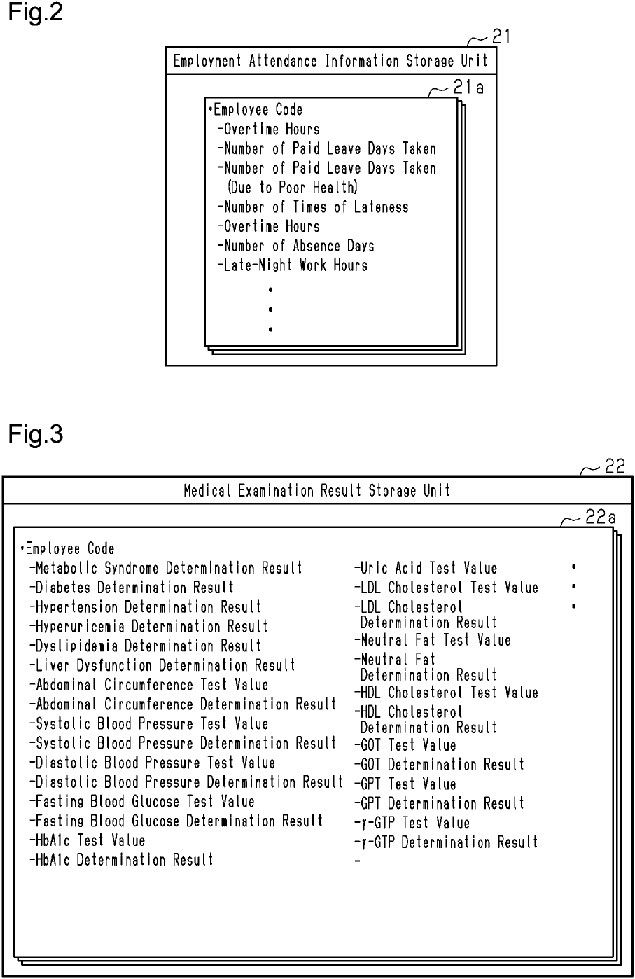

The employment attendance information storage unit 21, the medical examination result storage unit 22, and the stress check result storage unit 23 included in the database 20 will now be described with reference to FIGS. 2 to 4.

As illustrated in FIG. 2, the employment attendance information storage unit 21 stores the employment attendance information record 21a. The employment attendance information record 21a includes item data that is data of items related to an employment status of each employee. The employment attendance information record 21a is associated with an employee code that is a code assigned to each employee. The employment attendance information record 21a stores, as item data of employment attendance information of an employee to whom the employment attendance information record 21a is assigned, overtime hours, the number of paid leave days taken, the number of times of lateness, overtime hours, the number of absence days, late-night work hours, total working hours, and the like. The item data on the employment status of each employee is input from an attendance management system used by the company. The item data on the employment status of each employee may be input from the user terminal 1 via the network 2 by the person in charge of labor management of the company.

As illustrated in FIG. 3, the medical examination result storage unit 22 stores a medical examination result record 22a. The medical examination result record 22a includes item data that is data of items related to the medical examination of each employee. The medical examination result record 22a is associated with an employee code. The medical examination result record 22a includes a measured value and a determination result for each item of the medical examination as item data. The item data of the items concerning the medical examination of each employee is captured by processing digital data obtained from a medical examination center where the medical examination was performed or the data printed on a paper medium is manually input by a person in charge of the company.

The medical examination result record 22a includes, as medical examination results, measured values of medical examination items such as abdominal circumference, body mass index (BMI) value, systolic blood pressure, diastolic blood pressure, and uric acid. The medical examination result record 22a includes blood measured values such as a fasting blood glucose (FPG) measured value, a hemoglobin A1c (HbA1c) (National Glycohemoglobin Standardization Program (NGSP)) measured value, a low density lipoprotein (LDL) cholesterol measured value, a high density lipoprotein (HDL) cholesterol value, a neutral fat measured value, a glutamic oxaloacetic transaminase (GOT) (aspartate transaminase (AST)) measured value, a glutamic pyruvic transaminase (GPT) (alanine transaminase (ALT)) measured value, and a γ-glutamyl transpeptidase (y-GTP) measured value.

The medical examination result record 22a includes a systolic blood pressure determination result, a diastolic blood pressure determination result, a fasting blood glucose level determination result, an HbA1c determination result, an HDL cholesterol determination result, a neutral fat determination result, a GOT determination result, a GPT determination result, and the like determined for each measured value in four levels of “A”, “B”, “C”, and “D” on the basis of the criteria determination table 301. Note that a determination result “E” indicates that treatment is being performed for the item regardless of the measured value. As for the determination result of each measured value, “A” indicates good health, and a result closer to “D” indicates poorer health.

The medical examination result record 22a includes a metabolic syndrome determination result, a diabetes determination result, a hypertension determination result, a hyperuricemia determination result, a dyslipidemia determination result, and a liver dysfunction determination result, which are determined in four levels of “A”, “B”, “C”, and “D” on the basis of the above medical examination determination results or measured values. Determination result “E” indicates that treatment is being performed for the item regardless of the measured value. As for the determination result of the lifestyle disease, “A” indicates that the possibility of corresponding to the lifestyle disease is low, and a result closer to “D” indicates that the possibility of corresponding to the lifestyle disease is higher. Although the metabolic syndrome is not strictly a lifestyle disease, in the present embodiment, the metabolic syndrome is treated as a lifestyle disease.

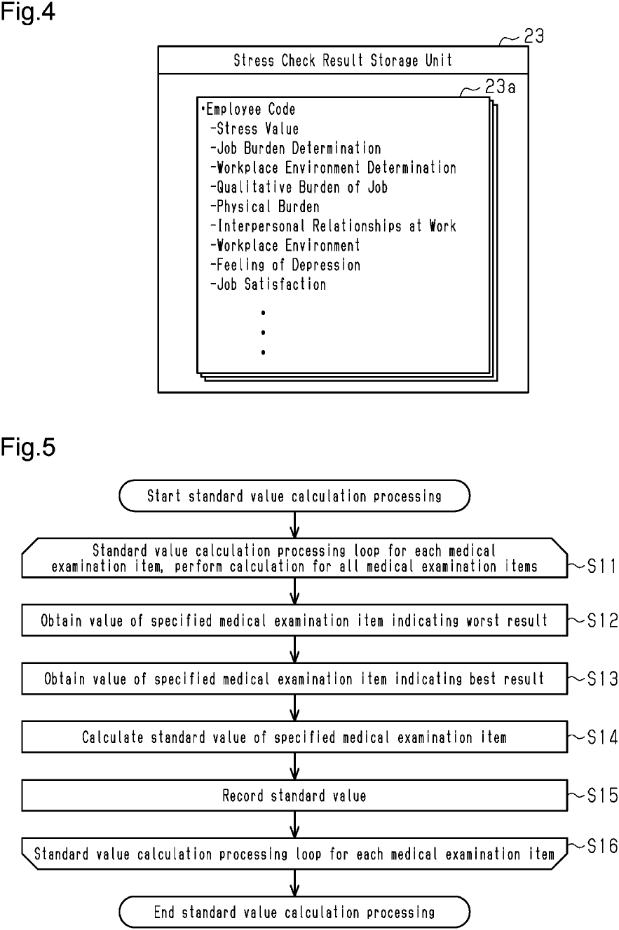

As illustrated in FIG. 4, the stress check result storage unit 23 stores the stress check result record 23a. The stress check result record 23a is associated with an employee code. The stress check result record 23a includes item data that is data of items concerning a stress check of each employee. The stress check result record 23a includes results such as a job burden determination, a workplace environment determination, a qualitative burden of the job, a physical burden, an interpersonal relationship at work, a workplace environment, a feeling of depression, and job satisfaction. Further, the stress check result record 23a includes an overall evaluation of five levels or two levels. The overall evaluation is a determination result of whether or not a person is a high-stress person, a person with a mental health disorder, or a person taking a leave of absence due to mental illness, obtained by calculating a total score of mental and physical stress reactions, job stress factors, and the like on the basis of each result included in the stress check result record 23a. Moreover, the stress check result record 23a includes a result of a stress value. The stress value is calculated using a known calculation method described in Patent Literature 2. The item data on the stress check is input by an industrial physician or the like who has performed the stress check.

### Calculation Processing

With reference to FIGS. 5 to 9, a series of procedures including the calculation of the absenteeism loss cost and the presenteeism loss cost and the output of the result to a screen or a file by the system for calculating the cost of labor productivity loss will now be described.

The person in charge of labor management operates the user terminal 1 to transmit an instruction to calculate the cost of labor productivity loss to the management server 10. The management server 10 that has received the calculation instruction calculates the cost of labor productivity loss in the labor productivity loss cost calculation unit.

### Standard Value Calculation Processing

As illustrated in FIG. 5, the standard value calculation unit 41 calculates a standard value to be used for processing of calculating a medical examination numerical value before the calculation of a labor productivity cost. The standard value is a normalization coefficient for calculating a medical examination numerical value and is a coefficient for using the difference between an upper limit value and a lower limit value that can be taken by the measured value of the medical examination item as “50”.

In step S11, the standard value calculation unit 41 starts standard value calculation processing for each medical examination item. In step S12, the standard value calculation unit 41 refers to the criteria determination table 301 of the specified medical examination item and obtains a lower limit value that can be taken by the measured value of the specified medical examination item. The lower limit value that can be taken by the measured value of the specified medical examination item is a numerical value corresponding to the urgent need for treatment in the criteria determination table 301 of the specified medical examination item. For example, when the specified medical examination item is the systolic blood pressure, the standard value calculation unit 41 refers to the systolic blood pressure criteria determination table 301a1 illustrated in FIG. 7 and obtains 160, which is a value listed in the D determination column.

In step S13, the standard value calculation unit 41 refers to the criteria determination table 301 of the specified medical examination item and obtains an upper limit value that can be taken by the measured value of the specified medical examination item. The upper limit value that can be taken by the measured value of the specified medical examination item is a numerical value corresponding to no abnormality in the criteria determination table 301 of the specified medical examination item. For example, when the specified medical examination item is the systolic blood pressure, the standard value calculation unit 41 refers to the systolic blood pressure criteria determination table 301a1 illustrated in FIG. 7 and obtains 129, which is a value listed in the A determination column.

In step S14, the standard value calculation unit 41 calculates the standard value of the specified medical examination item. As a calculation example of the standard value, the standard value is calculated by Equation 7, which is shown below, using the values obtained in steps S12 and S13.

$$\text{Standard value =} \left(\text{Lower limit value - Upper limit value}\right) / 50$$

Here, “50” is used as an intermediate value when the full score is 100. For example, the standard value of the systolic blood pressure calculated through Equation 7 is calculated as follows.

$$\left(\text{160-129}\right) / 50 = 0.62$$

In step S15, the standard value calculation unit 41 stores the calculated standard value into a systolic blood pressure criteria determination table 301b1 of the medical examination item that corresponds to the standard value. In step S16, when there remains a medical examination item for which the standard value calculation processing has not been ended, the standard value calculation unit 41 proceeds to step S11 and starts the standard value calculation processing for the next medical examination item.

For example, when the medical examination item specified next is the diastolic blood pressure, the standard value calculation unit 41 uses a diastolic blood pressure criteria determination table 301a2 illustrated in FIG. 7 to obtain 100, which is a value listed in the D determination column, as the lower limit value of the medical examination numerical value (step S12). Then, the standard value calculation unit 41 obtains 84, which is a value listed in the A determination column, as the upper limit value of the medical examination numerical value (step S13). Next, the standard value calculation unit 41 calculates the reference value of the diastolic blood pressure as follows (step S14).

$$\left(\text{100-84}\right) / 50 = 0.32$$

The standard value calculation unit 41 stores the calculated reference value into the reference value item of a diastolic blood pressure criteria determination table 301b2 (step S15). Then, when the standard value calculation processing is ended for all the medical examination items, the standard value calculation unit 41 ends the standard value calculation processing.

### Medical Examination Numerical Value Calculation Processing

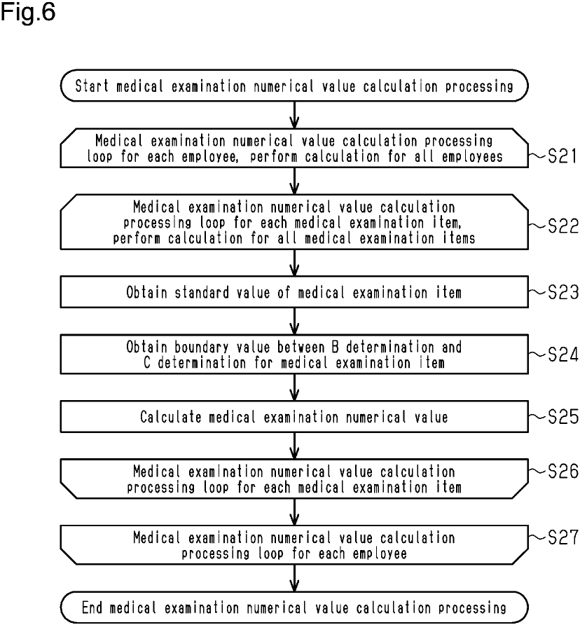

As illustrated in FIG. 6, the medical examination numerical value calculation unit 42 calculates the medical examination numerical values of all the target medical examination items for all the employees subject to the calculation of the cost of labor productivity loss. Here, the target medical examination items are abdominal circumference, BMI, systolic blood pressure, diastolic blood pressure, HDL cholesterol, LDL cholesterol, neutral fat, fasting blood glucose, HbA1c, uric acid, GOT, GPT, and γ-GTP.

In step S21, the medical examination numerical value calculation unit 42 starts the medical examination numerical value calculation processing for each employee. In step S22, the medical examination numerical value calculation unit 42 starts the medical examination numerical value calculation processing on each medical examination item for each employee.

In step S23, the medical examination numerical value calculation unit 42 refers to the criteria determination table 301 for each medical examination item and obtains the standard value of the target medical examination item calculated earlier. For example, when the target medical examination item is the systolic blood pressure, the medical examination numerical value calculation unit 42 refers to the standard value of the systolic blood pressure criteria determination table 301b1 and obtains 0.62 as the standard value of the systolic blood pressure.

In step S24, the medical examination numerical value calculation unit 42 refers to the criteria determination table 301 for each medical examination item and obtains the boundary value between the B determination and the C determination for the target medical examination item. For example, when the target medical examination item is the systolic blood pressure, the medical examination numerical value calculation unit 42 uses the systolic blood pressure criteria determination table 301b1 to obtain 139 that is the boundary value between the B determination and the C determination.

In step S25, the medical examination numerical value calculation unit 42 obtains the measured value of the target medical examination item of the employee corresponding to a person subject to the calculation from the medical examination result storage unit 22 and calculates the medical examination numerical value through Equation 8, which is shown below.

$$\begin{matrix}\text{Medical examination numerical value =} \\ \text{100 +} \left(\text{Boundary value between B}\right) \\ \text{determination and C determination -} \\ \text{Measured} \left(\text{value}\right) / \text{Standard value}\end{matrix}$$

That is, the medical examination numerical value is a numerical value indicating the size of the separation of the measured value from the boundary value between the B determination and the C determination, assuming that the difference between the lower limit value and the upper limit value is 50. The medical examination numerical value indicates better health as its value becomes larger from 100 and poorer health as its value becomes smaller from 100.

For example, when the medical examination item is systolic blood pressure and the measured value in the medical examination is 160, the following value is calculated as the medical examination numerical value with Equation 8, which is shown above (rounded to the second decimal place).

$$100 + \left(\text{139-160}\right) / 0.62 = 66.13$$

In step S26, the medical examination numerical value calculation unit 42 stores the calculated medical examination numerical value into the medical examination result storage unit 22. In step S27, the medical examination numerical value calculation unit 42 ends the medical examination numerical value calculation processing for each medical examination item. When there remains a medical examination item for which the medical examination numerical value calculation processing has not been ended for each employee, the medical examination numerical value calculation unit 42 proceeds to step S22 and starts the medical examination numerical value calculation processing in the next medical examination item.

For example, when the medical examination item specified next is the diastolic blood pressure, the medical examination numerical value calculation unit 42 refers to the diastolic blood pressure criteria determination table 301b2 and obtains a value of 0.32 as the standard value of the diastolic blood pressure (step S23). Subsequently, the medical examination numerical value calculation unit 42 refers to the diastolic blood pressure criteria determination table 301b2 and obtains a value of 89 as the boundary value between the B determination and the C determination for the diastolic blood pressure (step S24). When the measured value of the diastolic blood pressure is 95, the medical examination numerical value calculation unit 42 calculates the medical examination numerical value of the diastolic blood pressure as follows (rounded to the second decimal place) (step S25).

$$100 + \left(\text{89-95}\right) / 0.32 = 84.38$$

The medical examination numerical value calculation unit 42 stores the calculated medical examination numerical value into the medical examination result storage unit 22 (step S26). Further, in step S27, when the medical examination numerical value calculation processing is ended for all the medical examination items for each employee, the medical examination numerical value calculation unit 42 ends the medical examination numerical value calculation processing for each employee. When there remains an employee for whom medical examination numerical value calculation processing has not been ended, the medical examination numerical value calculation unit 42 proceeds to step S21 and starts the medical examination numerical value calculation processing for the next employee.

When the medical examination numerical value calculation processing is ended for all the employees subject to the calculation of the cost of labor productivity loss, the medical examination numerical value calculation unit 42 ends the medical examination numerical value calculation processing.

### Stress Loss Cost Calculation Processing

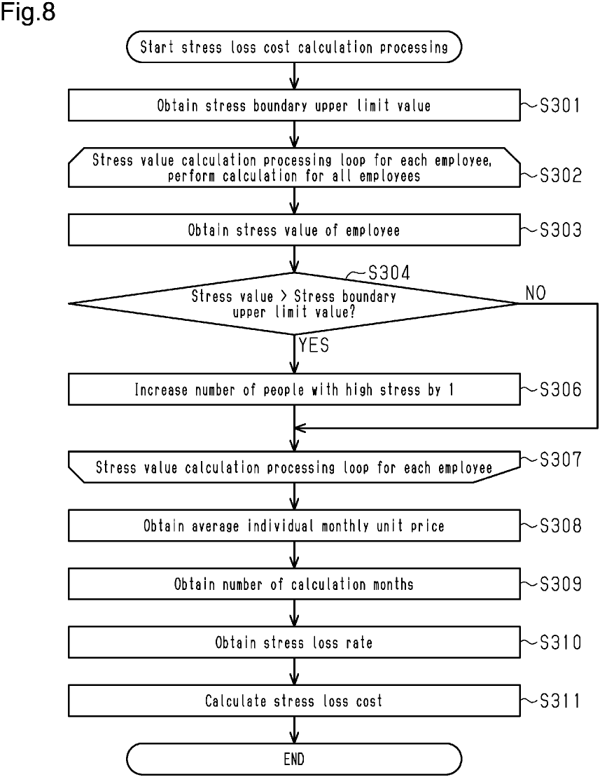

As illustrated in FIG. 8, the stress loss cost calculation unit 43 calculates a stress loss cost from stress values of all the employees subject to the calculation of the cost of labor productivity loss. In step S301, the stress loss cost calculation unit 43 obtains the stress boundary upper limit value 306 from the setting data storage unit 300. In step S302, the stress loss cost calculation unit 43 starts stress loss cost calculation processing for each employee.

In step S303, the stress loss cost calculation unit 43 extracts the stress value of the employee from the stress check result storage unit 23. In step S304, the stress loss cost calculation unit 43 determines whether or not the stress value of the employee exceeds the stress boundary upper limit value 306.

When the stress value of the employee exceeds the stress boundary upper limit value 306 (YES in step S304), 1 is added to the number of people with high stress, which is a program variable in step S306. That is, when an employee having a stress value exceeding the stress boundary upper limit value 306 is defined as a person with high stress, the number of people with high stress in the company is the number of people with high stress.

In step S307, when there remains an employee for whom the determination of the stress value has been ended, the stress loss cost calculation unit 43 proceeds to step S302 and determines the stress value for the remaining employees. When the determination of the stress values is ended for all the employees subject to the calculation of the cost of labor productivity loss, the stress loss cost calculation unit 43 proceeds to step S308.

In step S308, the stress loss cost calculation unit 43 obtains the average individual monthly unit price 303 from the setting data storage unit 300. In step S309, the stress loss cost calculation unit 43 obtains the number of labor productivity loss cost calculation months 307 from the setting data storage unit 300. In step S310, the stress loss cost calculation unit 43 obtains the stress loss rate 304 from the setting data storage unit 300. In step S311, the stress loss cost calculation unit 43 calculates the stress loss cost with Equation 3, which is shown below.

$$\begin{matrix}\text{Stress loss cost =} \\ \text{Number of people with high stress} \times \\ \text{Number of labor productivity} \\ \text{loss cost calculation months} \times \\ \text{Average individual monthly unit price} \times \text{Stress loss rate}\end{matrix}$$

Lifestyle disease loss cost calculation processing

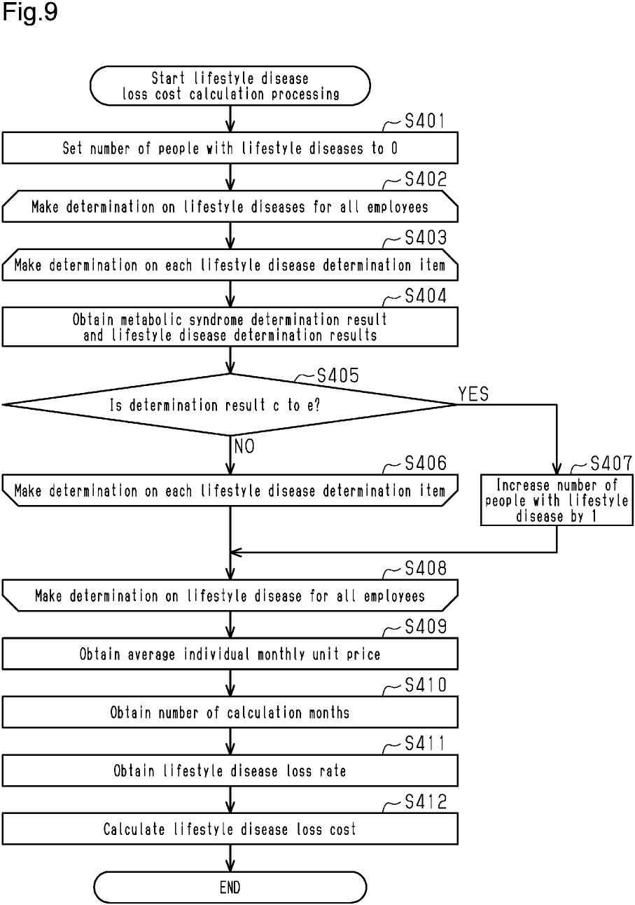

As illustrated in FIG. 9, the lifestyle disease loss cost calculation unit 44 performs metabolic syndrome determination and lifestyle disease determination provided as medical examination results for all the employees subject to the calculation of the cost of labor productivity loss and calculates a lifestyle disease loss cost.

In steps S401 to S412, the lifestyle disease loss cost calculation unit 44 determines whether or not each employee is a person with a lifestyle disease on the basis of lifestyle disease determination items for each employee, specifically, the metabolic syndrome determination result, the diabetes determination result, the hypertension determination result, the hyperuricemia determination result, the dyslipidemia determination result, and the liver dysfunction determination result, and calculates the lifestyle disease loss cost for the entire company.

In step S401, the number of people with a lifestyle disease is set to 0. In step S402, the determination as to whether or not each employee is a person with a lifestyle disease is started. In step S403, it is determined whether or not each of a plurality of lifestyle disease determination items described above corresponds to the C to E determinations for one employee.

In step S404, the lifestyle disease determination item, specifically, the metabolic syndrome determination result, the diabetes determination result, the hypertension determination result, the hyperuricemia determination result, the dyslipidemia determination result, and the liver dysfunction determination result are sequentially obtained from the medical examination result storage unit 22.

In step S405, it is determined whether or not the determination result of the lifestyle disease determination item is “C” to “E”. When the metabolic syndrome determination result, the diabetes determination result, the hypertension determination result, the hyperuricemia determination result, the dyslipidemia determination result, or the liver dysfunction determination result of the employee is the C determination, the D determination, or the E determination (YES in step S405), the processing proceeds to step S407. When the metabolic syndrome determination result, the diabetes determination result, the hypertension determination result, the hyperuricemia determination result, the dyslipidemia determination result, or the liver dysfunction determination result of the employee is not the C determination, the D determination, or the E determination (NO in step S405), the processing proceeds to step S406.

In step S407, 1 is added to the number of people with a lifestyle disease. That is, when an employee having a lifestyle disease with the lifestyle disease determination of “C” to “E” is defined as a person with a lifestyle disease, the number of people with a lifestyle disease in the company is the number of people with a lifestyle disease. Then, the lifestyle disease loss cost calculation unit 44 proceeds to step S408.

In step S406, when there remains a lifestyle disease determination item for which the checking of the determination result has not been performed, the lifestyle disease loss cost calculation unit 44 proceeds to step S403 and starts checking the remaining lifestyle disease determination items. When the checking of all the lifestyle disease determination items is ended, the lifestyle disease loss cost calculation unit 44 proceeds to step S408.

In step S408, when there remains an employee for whom the determination as to whether or not the employee is a person with a lifestyle disease has not been ended, the lifestyle disease loss cost calculation unit 44 proceeds to step S402 and starts the determination as to whether or not the remaining employee is a person with a lifestyle disease. When the determination as to whether or not all the employees subject to the calculation of the cost of labor productivity loss are people with lifestyle diseases is ended, the lifestyle disease loss cost calculation unit 44 proceeds to step S409.

In step S409, the average individual monthly unit price 303 is obtained from the setting data storage unit 300. In step S410, the number of labor productivity loss cost calculation months 307 is obtained from the setting data storage unit 300. In step S411, the lifestyle disease loss rate 305 is obtained from the setting data storage unit 300. In step S412, the lifestyle disease loss cost is calculated with Equation 4, which is shown below.

$$\begin{matrix}\text{Lifestyle disease loss cost =} \\ \text{Number of people with a lifestyle disease} \times \text{Average} \\ \text{individual monthly unit price} \times \\ \text{Number of labor productivity loss calculation months} \\ \text{Lifestyle disease loss rate}\end{matrix}$$

### Absenteeism Loss Cost Calculation Processing

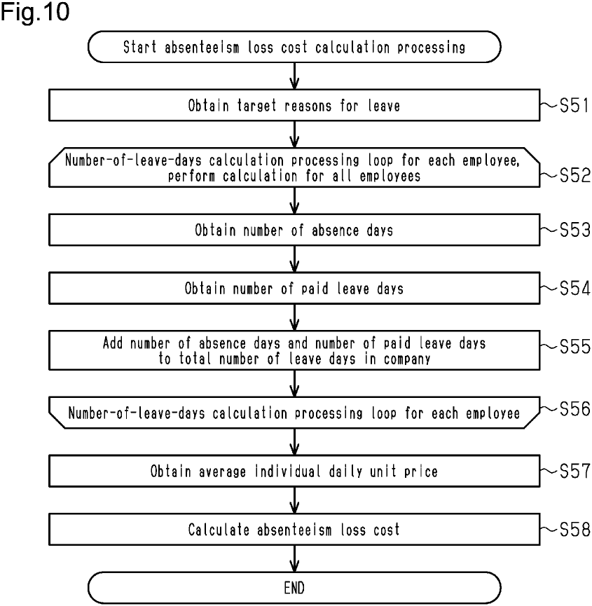

As illustrated in FIG. 10, the absenteeism loss cost calculation unit 45 calculates an absenteeism loss cost. Steps S51 to S58 are performed on all the employees subject to the calculation of the cost of labor productivity loss.

In step S51, the target reasons for leave 308 are obtained from the setting data storage unit 300. In step S52, the absenteeism loss cost calculation unit 45 starts processing of calculating the number of leave days of each employee in a period for which the cost of labor productivity loss is calculated. In step S53, the employment attendance information storage unit 21 is referred to, and the number of absence days of the employee is obtained. In step S54, the employment attendance information storage unit 21 is referred to, and the number of paid leave days taken due to reasons for leave corresponding to the target reasons for leave 308 for the employee is obtained. In step S55, the number of absence days obtained in step S53 and the number of paid leave days obtained in step S54 are added to the total number of leave days from the company, which is a program variable.

In step S56, when there remains an employee for whom the processing of calculating the number of leave days has not been ended, the absenteeism loss cost calculation unit 45 proceeds to step S52 and starts the processing of calculating the number of leave days for the next employee. When the processing of calculating the number of leave days is ended for all the employees subject to the calculation of the cost of labor productivity loss, the absenteeism loss cost calculation unit 45 proceeds to step S57.

In step S57, the average individual daily unit price 302 is obtained from the setting data storage unit 300. In step S58, the absenteeism loss cost is calculated using the following equation.

$$\begin{matrix}\text{Absenteeism loss cost =} \\ \text{Total number of leave days from company} \times \text{Average} \\ \text{individual daily unit price}\end{matrix}$$

Finally, the labor productivity loss cost calculation unit 40 calculates the cost of labor productivity loss with Equations 1 and 2, which are shown below. At this time, with reference to the employment attendance information record 21a, the overtime hours of all the employees in the calculation period are obtained, and the average individual hourly unit price is calculated from the average individual daily unit price 302 to calculate an overtime loss cost.

$$\begin{matrix}\text{Presenteeism loss cost =} \\ \text{Lifestyle disease loss cost + Stress loss cost}\end{matrix}$$

$$\begin{matrix}\text{Cost of labor productivity loss =} \\ \text{Absenteeism loss cost + Presenteeism loss cost +} \\ \text{Overtime loss cost}\end{matrix}$$

The management server 10 outputs data including the calculated cost of labor productivity loss from the screen output unit 51 to the user terminal 1 via the network 2. The data including the cost of labor productivity loss is, for example, a tabular format storing a history of the cost of labor productivity loss for each year, or data representing a change in the cost of labor productivity loss for each year in a graphical format. The user terminal 1 outputs data including the cost of labor productivity loss output from the screen output unit 51 by the output unit included in the user terminal 1. For example, the user terminal 1 outputs the cost of labor productivity loss by displaying the data in the tabular format or the graphical format on the display.

Further, the management server 10 outputs data including the calculated cost of labor productivity loss from the form output unit 52 to a printer (not illustrated) via the network 2. The printer prints the output data including the cost of labor productivity loss and outputs the data as a form.

The embodiment described above has the following advantages. (1) The system for calculating the cost of labor productivity loss calculates the presenteeism loss cost from the item data of the medical examination result record 22a stored in the medical examination result storage unit 22 and the item data of the stress check result record 23a stored in the stress check result storage unit 23. Therefore, even when there is an error resulting from uncertain memory of an employee included in answers to a questionnaire such as whether or not the employee is at work with a disease or a symptom or an error caused by ambiguous evaluation depending on an individual determination criterion, the influence of such an error is reduced by adding accurate data such as a medical examination result. As a result, it is possible to improve the accuracy for the calculation of the cost of labor productivity loss.

(2) The system for calculating the cost of labor productivity loss calculates the cost of labor productivity loss with Equations 1 and 2, which are shown above, so that the cost of labor productivity loss can be calculated taking into consideration the decrease in labor productivity caused by the mental health disorder in addition to the decrease in labor productivity caused by the physical health disorder of the employee.

(3) The system for calculating the cost of labor productivity loss calculates the stress loss cost with Equation 3 above so that the stress loss cost corresponding to the number of people with high stress can be calculated. (4) The system for calculating the cost of labor productivity loss calculates the lifestyle disease loss cost with Equation 4, which is shown above, so that the lifestyle disease loss cost corresponding to the number of people with a lifestyle disease can be calculated.

(5) The system for calculating the cost of labor productivity loss calculates the absenteeism loss cost with Equation 5, which is shown above, so that the cost of labor productivity loss can be calculated without performing an additional questionnaire to calculate the absenteeism loss cost.

### Second Embodiment

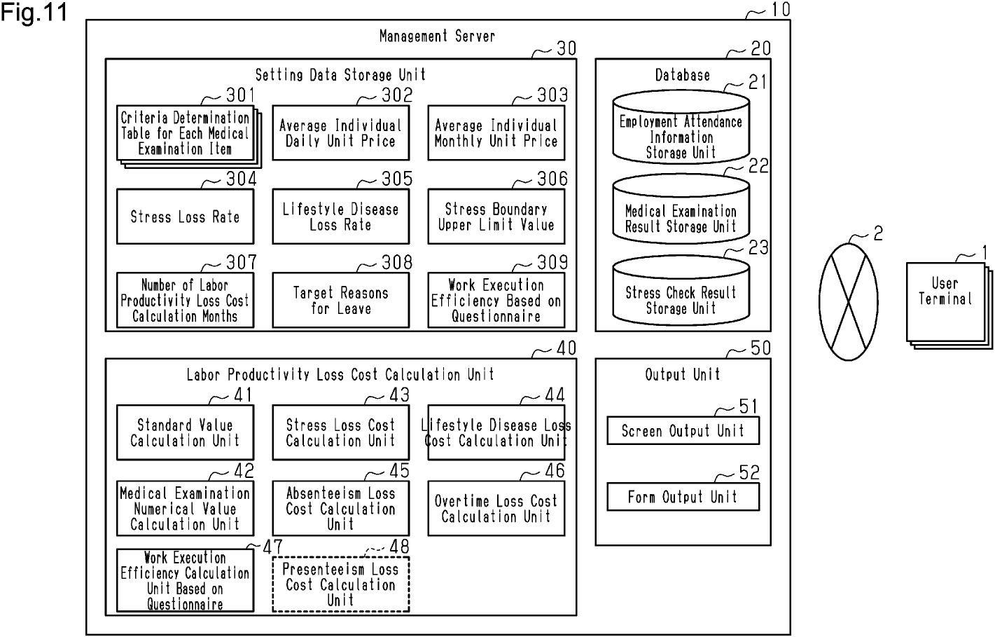

A second embodiment of a system for calculating cost of labor productivity loss, a program for calculating cost of labor productivity loss, and a method for calculating cost of labor productivity loss will now be described with reference to FIGS. 11 to 14. The system for calculating the cost of labor productivity loss of the second embodiment differs from the first embodiment in that results of a questionnaire on work execution efficiency are stored.

As illustrated in FIG. 11, the setting data storage unit 300 further stores work execution efficiency 309 based on a questionnaire. The labor productivity loss cost calculation unit 40 further includes a work execution efficiency calculation unit 47 based on the questionnaire.

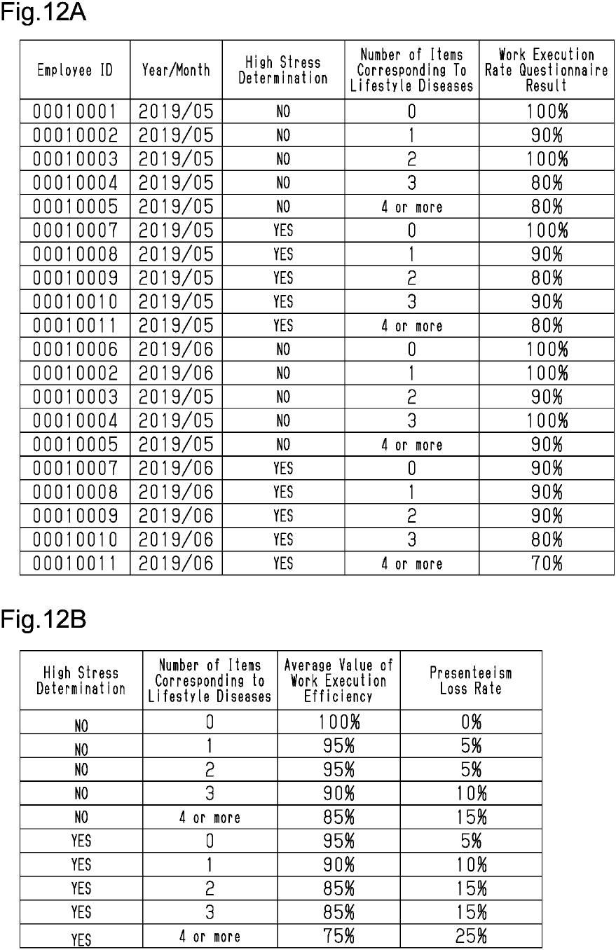

As illustrated in FIG. 12A, the work execution efficiency 309 of each employee based on the questionnaire stores work execution efficiency data answered by each employee in response to questionnaire questions made by an industrial physician or a public health nurse in association with an employee code and an answer date. The work execution efficiency is obtained from each employee who compares the work execution ability and the productivity before any disease or symptom occurs that is set to 100% with the work execution ability and the productivity efficiency in a predetermined period. Further, the work execution efficiency 309 based on the questionnaire stores a high stress determination result and the number of items corresponding to the lifestyle diseases together with the work execution efficiency data answered by each employee. The number of items corresponding to the lifestyle diseases is the number of lifestyle diseases determination items corresponding to the C to E determinations among the lifestyle diseases determination items for each employee.

In the first embodiment described above, the stress loss rate and the lifestyle disease loss rate are the system eigenvalues or set by the user from the user terminal 1. In the second embodiment, the stress loss rate and the lifestyle disease loss rate are calculated from the result of the questionnaire to the employee at the time of face-to-face guidance or health guidance by the industrial physician or the public health nurse, and are used when the stress loss cost and the lifestyle disease loss cost are calculated.

The questionnaire is conducted by the industrial physician or the public health nurse on employees at the time of face-to-face guidance or health guidance, and the questionnaire result is input from the user terminal 1 via a questionnaire result input screen provided by the management server 10 and stored into the work execution efficiency 309 based on the questionnaire in association with the employee code of each employee.

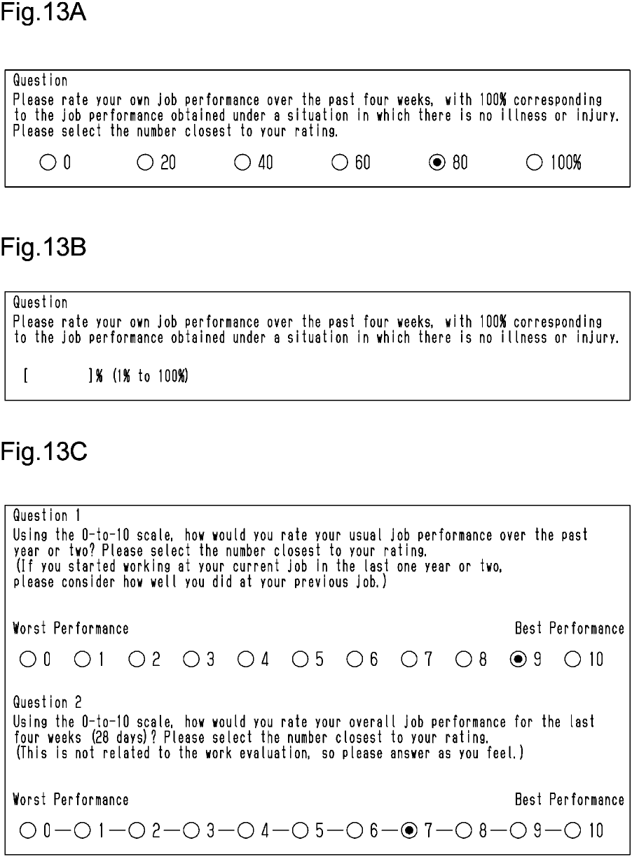

The questionnaire contains questions such as those shown in FIGS. 13A, 13B, and 13C. For example, in FIG. 13A, when 100% corresponds to the job performance obtained under a situation in which there is no illness or injury, the job performance of the past four weeks at the time of the face-to-face guidance or the health guidance is selected from the six ratings of 0%, 20%, 40%, 60%, 80%, and 100%.

Further, for example, FIG. 13B has a format referred to as the University of Tokyo, Single-Item Presenteeism Question, and questions having substantially the same contents as FIG. 13A are given to the subject. The difference from FIG. 13A is that each subject evaluates the work execution efficiency by oneself with a rating of 1% to 100%, and that this self-rating may be used.

Moreover, for example, FIG. 13C shows questions B10 and B11 of the World Health Organization Health and Work Performance Questionnaire Short Form (WHO-HPQ Short Form), which may be used as a questionnaire. As the work execution efficiency of the individual at this time, the ratio of Questions 1 and 2 of FIG. 13C can be adopted.

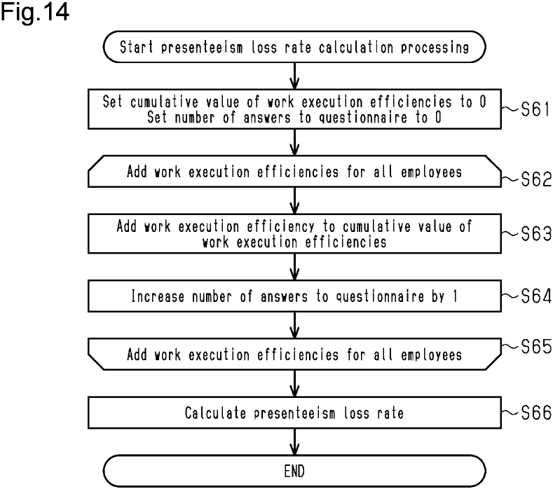

As illustrated in FIG. 14, the work execution efficiency calculation unit 47 based on the questionnaire obtains the work execution efficiency 309 based on the questionnaire and calculates a presenteeism loss rate from the work execution efficiency 309 based on the questionnaire as the same value as that applied to the stress loss rate and the lifestyle disease loss rate. Steps S61 to S66 are performed on all the questionnaire results answered in a specified period.

In steps S61 to S66, the work execution efficiency calculation unit 47 based on the questionnaire calculates the presenteeism loss rate on the basis of the questionnaire result answered in the specified period.

In step S61, a cumulative value of work execution efficiencies and the number of answers to the questionnaire are set to 0. In step S62, counting of the work execution efficiencies in the questionnaire results answered in the specified period is started. In step S63, the value of the work execution efficiency obtained from the obtained questionnaire result is added to the cumulative value of work execution efficiencies. In step S64, 1 is added to the number of answers to the questionnaire.

In step S65, when there remains a questionnaire result to which the work execution efficiency has not been added among the questionnaire results answered in the specified period, the work execution efficiency calculation unit 47 based on the questionnaire proceeds to step S62 and continues to count the questionnaire results. When the aggregation of the questionnaire results is ended, the work execution efficiency calculation unit 47 based on the questionnaire proceeds to step S66.

In step S66, the presenteeism loss rate is calculated by subtracting a numerical value obtained by dividing the cumulative value of work execution efficiencies by the number of answers to the questionnaire from 100%. That is, the presenteeism loss rate is expressed by Equation 8, which is shown below. Presenteeism loss rate = 100% - cumulative value of work execution efficiencies/Number of answers to questionnaire ··· (Equation 8)

The labor productivity loss cost calculation unit 40 stores the presenteeism loss rate obtained here into the stress loss rate 304 and the lifestyle disease loss rate 305. The labor productivity loss cost calculation unit 40 calculates the cost of labor productivity loss by using the stored stress loss rate 304 and the lifestyle disease loss rate 305.

### Third Embodiment

A third embodiment, which is a modified example of the second embodiment, will now be described with reference to FIGS. 11 to 13 and 15. The system for calculating the cost of labor productivity loss of the third embodiment differs from the second embodiment in that a labor productivity loss cost calculation unit 40 includes a presenteeism loss cost calculation unit 48. Further, this system different from the second embodiment in that the work execution efficiency of the questionnaire result is classified in accordance with the stress value of the employee who has answered the questionnaire and the result of the lifestyle disease determination item, and on the basis of the work execution efficiency, the presenteeism loss cost calculation unit 48 calculates the cost of labor productivity loss corresponding to the stress value of the employee and the result of the lifestyle disease determination item.

As illustrated in FIG. 12B, the work execution efficiency 309 based on the questionnaire further includes a combination table in which the average value of the work execution efficiency and the presenteeism loss rate are associated with each combination of the result of the high stress determination and the number of items corresponding to the lifestyle diseases.

The average value of the work execution efficiency associated with the combination of the result of the high stress determination and the number of items corresponding to the lifestyle diseases is an average value of the work execution efficiency in the extracted questionnaire results when the questionnaire results in which the result of the high stress determination and the number of items corresponding to the lifestyle diseases match are extracted from the questionnaire results illustrated in FIG. 12A. The presenteeism loss rate associated with the combination of the result of the high stress determination and the number of items corresponding to the lifestyle diseases is a numerical value obtained by subtracting the average value of the work execution efficiency from 100%.

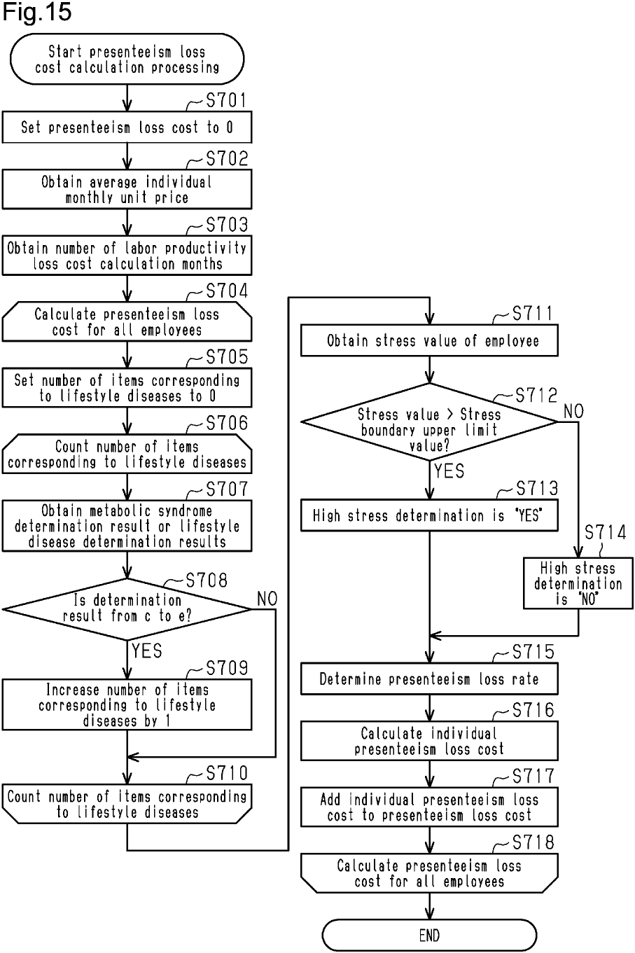

The presenteeism loss cost calculation unit 48 calculates the presenteeism loss cost on the basis of the combination table. The presenteeism loss cost in this case is calculated by the processing illustrated in FIG. 15 instead of Equation (8), which is shown above.

The presenteeism loss cost calculation unit 48 calculates the presenteeism loss cost of the entire company through steps S701 to S717.

In step S701, the presenteeism loss cost of the entire company is set to 0. In step S702, the average individual monthly unit price 303 is obtained from the setting data storage unit 300. In step S703, the number of labor productivity loss cost calculation months 307 is obtained from the setting data storage unit 300.

In step S704, processing of calculating the presenteeism loss cost for each employee and the presenteeism loss cost of the entire company is started. In step S705, the number of items corresponding to the lifestyle diseases is set to 0. In step S706, it is determined whether or not the lifestyle disease determination result corresponds to a specific determination result for one employee.

In step S707, the lifestyle disease determination item, specifically, the metabolic syndrome determination result, the diabetes determination result, the hypertension determination result, the hyperuricemia determination result, the dyslipidemia determination result, and the liver dysfunction determination result are sequentially obtained from the medical examination result storage unit 22. In step S708, it is determined whether or not the value of the lifestyle disease determination item is from “C” to “E”. When the metabolic syndrome determination result, the diabetes determination result, the hypertension determination result, the hyperuricemia determination result, the dyslipidemia determination result, or the liver dysfunction determination result of the employee is the C determination, the D determination, or the E determination, the processing proceeds to step S709.

In step S709, 1 is added to the number of items corresponding to the lifestyle diseases. In step S710, when there remains a lifestyle disease determination item for which the checking of the lifestyle disease determination result has not been performed, the processing proceeds to step S706, and the checking of the remaining lifestyle disease determination items is started. When the checking of all the lifestyle disease determination items is ended, the processing proceeds to step S711.

In step S711, the stress value of the employee is extracted from the stress check result storage unit 23. In step S712, it is determined whether or not the stress value of the employee exceeds the stress boundary upper limit value 306.

When the stress value of the employee exceeds the stress boundary upper limit value 306 (YES in step S712), the processing proceeds to step S713. In step S713, the result of the high stress determination of the employee is “Yes”, and the processing proceeds to step S715. When the stress value of the employee is less than or equal to the stress boundary upper limit value 306 (NO in step S712), the processing proceeds to step S714. In step S714, the result of the high stress determination of the employee is “No”, and the processing proceeds to step S715.

In step S715, from the table of FIG. 12B, the presenteeism loss rate is obtained from the row in which the result of the high stress determination and the number of items corresponding to the lifestyle diseases match. In step S716, the individual presenteeism loss cost is calculated with Equation 9, which is shown below.

$$\begin{matrix}\text{Individual presenteeism loss cost =} \\ \text{Presenteeism loss rate} \times \text{Number of labor} \\ \text{productivity loss cost calculation months} \times \\ \text{Average individual monthly unit price}\end{matrix}$$

In step S716, the individual presenteeism loss cost is added to the presenteeism loss cost of the entire company. That is, the presenteeism loss cost of the entire company is the sum of the individual presenteeism loss costs of all the employees.

In step S717, when there remains an employee for whom the calculation of the individual presenteeism loss cost has not been ended, the processing proceeds to step S704, and the calculation of the individual presenteeism loss cost is started for the remaining employees. When the calculation of the individual presenteeism loss cost is ended for all the employees subject to the calculation of the cost of labor productivity loss, the presenteeism loss cost calculation unit 48 ends the processing.

### Fourth Embodiment

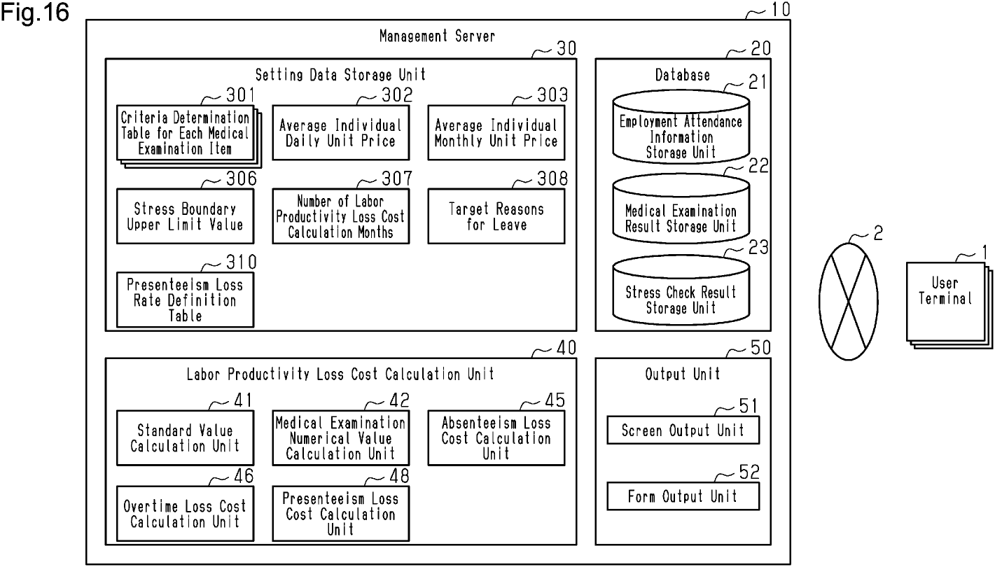

A fourth embodiment, which is a modified example of the third embodiment, will now be described with reference to FIGS. 16 to 19. The system for calculating the cost of labor productivity loss of the fourth embodiment does not use the stress loss rate 304, the stress loss cost calculation unit 43, the lifestyle disease loss rate 305, and the lifestyle disease loss cost calculation unit 44 described with reference to FIG. 11 and treats the stress loss as being the same as the lifestyle disease loss. Then, in the system for calculating the cost of labor productivity loss according to the fourth embodiment, the presenteeism loss cost calculation unit 48 calculates the presenteeism loss cost on the basis of the fact that the stress loss is the same as the lifestyle disease loss.

The setting data storage unit 300 may store the stress loss rate 304 and the lifestyle disease loss rate 305 but does not have to. The labor productivity loss cost calculation unit 40 may include the stress loss cost calculation unit 43 and the lifestyle disease loss cost calculation unit 44 but does not have to.

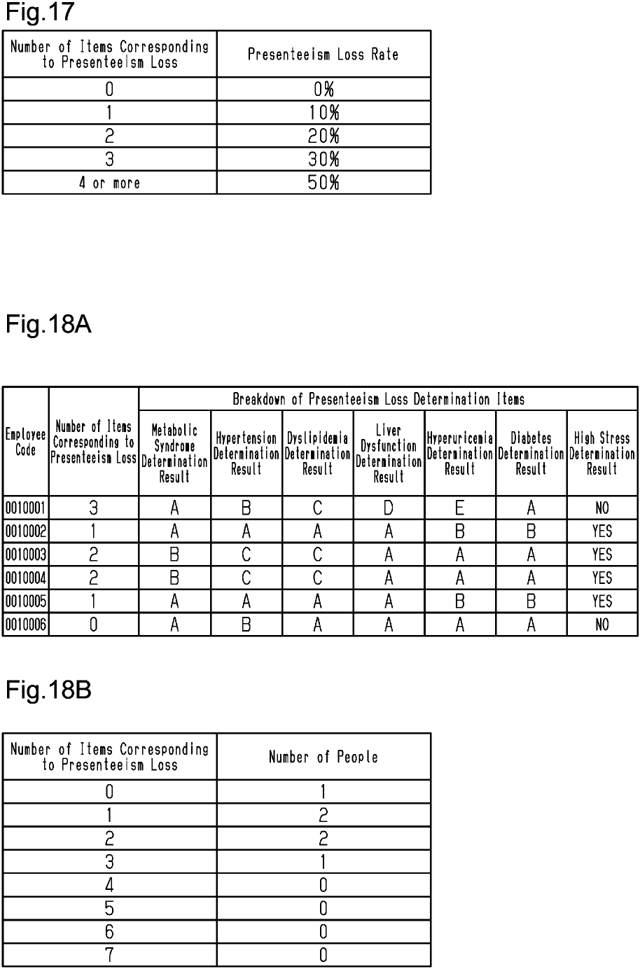

As illustrated in FIG. 17, the setting data storage unit 300 stores a presenteeism loss rate definition table 310. The presenteeism loss rate definition table 310 is a combination table in which the number of items corresponding to the presenteeism loss, which is the number of items corresponding to the criteria among the presenteeism determination items, is associated with the presenteeism loss rate.

The presenteeism determination item is an item for determining, for each employee, whether or not the presenteeism loss cost has been generated and is, for example, a determination result of metabolic syndrome, a determination result of each lifestyle disease, and a high stress determination result. The determination result of metabolic syndrome and the determination result of each lifestyle disease are counted as the number of items corresponding to the presenteeism loss when, with the determination results of C to E being used as criteria, the determination results meet the criteria. The high stress determination result is counted as the number of items corresponding to the presenteeism loss on the basis of the fact that the stress value exceeds the stress upper limit boundary value when the stress value meets the criterion.

In the presenteeism loss rate definition table 310, a presenteeism loss rate preset by the company may be stored as the presenteeism loss rate, or a presenteeism loss rate calculated in a procedure using the questionnaire illustrated in FIGS. 13 and 14 may be stored.

In the form in which the presenteeism loss rate based on the questionnaire is used, when the “high stress determination” in FIG. 12B is “Yes”, 1 is added to the “number of items corresponding to the lifestyle diseases”, and when the “high stress determination” is “No”, 1 is not added to the “number of items corresponding to the lifestyle diseases”. For example, in FIG. 12B, the presenteeism loss rate when the “high stress determination” is “Yes” and the “number of items corresponding to the lifestyle diseases” is “2” is set to 15%. This “15%” is used as the presenteeism loss rate when the number of items corresponding to the presenteeism loss is “3 (= 2 + 1)” in FIG. 18A.

The presenteeism loss cost calculation unit 48 checks the number of items corresponding to the presenteeism loss, which corresponds to the criterion, among the presenteeism loss determination items for each employee from the data of the medical examination result storage unit 22 and the data of the stress check result storage unit 23 for all the employees.

For example, as illustrated in FIG. 18A, a breakdown of the presenteeism loss determination items for each employee is configured as an example. In the data search results of the employee code “0010001” from the medical examination result storage unit 22 and the stress check result storage unit 23, the metabolic syndrome determination result is A, the hypertension determination result is B, the dyslipidemia determination result is C, the diabetes determination result is A, the liver dysfunction determination result is D, the hyperuricemia determination result is E, and the stress determination result is “No”. Thus, the number of items corresponding to the presenteeism loss among the presenteeism loss determination items for the employee code “0010001” is 3.

The presenteeism loss cost calculation unit 48 reads the loss rate that corresponds to each number of items corresponding to the presenteeism loss from the combination table in FIG. 17. Then, the presenteeism loss cost calculation unit 48 calculates the presenteeism loss cost of the entire employees by using the number of people for each number of items corresponding to the presenteeism loss, the loss rate that corresponds to the number of items corresponding to the presenteeism loss, the number of calculation months, and the average individual monthly unit price.

In this case, Equation 10, which is shown below, is used as the presenteeism loss cost instead of Equations 1, 3, and 4. Presenteeism loss cost

$$\begin{matrix}= {\sum }_{i = 1}^{7} \left(\text{Loss rate of number of items corresponding}\right) \\ \text{to presenteeism of i} \\ \times \text{Number of people who correspond to number of items} \\ \text{corresponding to presenteeism loss of i} \times \\ \text{Number of calculation} \\ \times \text{Average individual monthly unit} \left(\text{price}\right)\end{matrix}$$

The minimum value of the number of items corresponding to the presenteeism loss is 0, and the maximum value is the same as the number of presenteeism determination items. In the present embodiment, the presenteeism loss cost of the employee having the number of presenteeism loss determinations of 0 is 0 and is thus excluded, and the presenteeism loss cost is calculated by the number of people who correspond to the number of presenteeism loss determination items of 1 to 7.

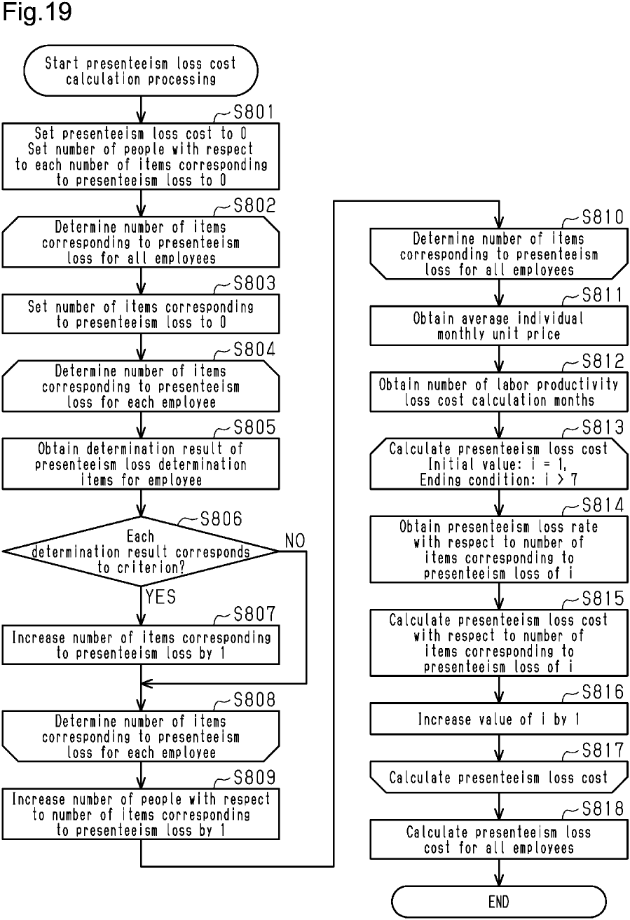

The calculation using Equation 10 will be described with reference to FIG. 19. The presenteeism loss cost calculation unit 48 calculates the presenteeism loss cost of the entire company through steps S801 to S817 in FIG. 19.

In step S801, the number of people who correspond to the number of items corresponding to the presenteeism loss of each of 0 to 7 is initialized and set to 0. In step S802, processing of determining the number of items corresponding to the presenteeism loss for all the employees is started. In step S803, the number of items corresponding to the presenteeism loss is set to 0. In step S804, processing of calculating the number of items corresponding to the presenteeism loss for one employee is started.

In step S805, one of the determination results of the presenteeism loss determination items for one employee is obtained from the data of the medical examination result storage unit 22 and the data of the stress check result storage unit 23.

In step S806, it is determined whether or not the obtained determination result of the presenteeism loss determination item corresponds to a criterion. The criteria of the metabolic syndrome determination result and each lifestyle disease determination result obtained from the medical examination result storage unit 22 are that the determination results are from C to E. The criterion of the high stress determination result obtained from the stress check result storage unit 23 is that the stress value exceeds the stress upper limit boundary value. When the presenteeism loss determination item meets the criterion (“Yes” in step S806), the processing proceeds to step S807. When the presenteeism loss determination item does not meet the criterion (in a case of “No” in step S806), the processing proceeds to step S808.

In step S807, 1 is added to the number of items corresponding to the presenteeism loss of the employee. Then, the processing proceeds to step S808. In step S808, the processing proceeds to step S804 when there is any remaining presenteeism determination item to be determined, and the processing proceeds to step S809 when there is no remaining presenteeism determination item.

In step S809, the number of people who correspond to the number of items corresponding to the presenteeism loss is increased by 1. For example, in the case of the employee code 0010001, since the number of items corresponding to the presenteeism loss is 3, the number of people who correspond to the number of items corresponding to the presenteeism loss of 3 is increased by one.

In step S810, when there remains an employee for whom the determination of the number of items corresponding to the presenteeism loss of the individual has not been ended, the processing proceeds to step S802, and processing of determining the number of items corresponding to the presenteeism loss of the individual for the remaining employees is started. When the determination of the number of items corresponding to the presenteeism loss of the individual is ended for all the employees subject to the calculation of the cost of labor productivity loss, the processing proceeds to step S811. At this time, as illustrated in FIG. 18B, data indicating the number of people with respect to each of the numbers of items corresponding to the presenteeism loss may be created and used for the calculation of the cost of labor productivity loss.

In step S811, the average individual monthly unit price 303 is obtained from the setting data storage unit 300. In step S812, the number of labor productivity loss cost calculation months 307 is obtained from the setting data storage unit 300.

In step S813, the presenteeism loss cost is calculated for each number of items corresponding to the presenteeism loss among the presenteeism loss determination items. That is, starting from the calculation of the presenteeism loss cost of the employee having the number of items corresponding to the presenteeism loss of 1, the presenteeism loss cost of the employee having the number of items corresponding to the presenteeism loss of 2 is calculated, followed by the calculation of the presenteeism loss cost of the employee having the number of items corresponding to the presenteeism loss of 3, and finally, the presenteeism loss cost of the employee having the presenteeism loss cost of 7 is calculated to end the calculation. This number of items corresponding to the presenteeism loss is defined as i.

In step S814, the presenteeism loss rate of the number of items corresponding to the presenteeism loss of i is obtained from the combination table of the presenteeism loss rates in FIG. 17. For example, when i is 1, 10%, which is a presenteeism loss rate in which the number of items corresponding to the presenteeism loss is 1, is obtained. In step S815, the presenteeism loss cost of the number of items corresponding to the presenteeism loss of i is calculated with the following equation.

$$\begin{matrix}\text{Presenteeism loss cost of number of items corresponding} \\ \text{to presenteeism loss of i =} \\ \text{Presenteeism loss rate of number of items corresponding} \\ \text{to presenteeism loss of i} \times \text{Number} \\ \\ \text{of people who corresponded to number of items corresponding} \\ \text{to presenteeism loss of i} \times \\ \text{Number of calculation months} \\ \times \text{Average individual monthly unit price}\end{matrix}$$

In step S816, i is increased by 1. In step S817, when there remains the next number of items corresponding to the presenteeism loss for calculating the presenteeism loss cost, that is, when i is 7 or less, the processing proceeds to step S813. When there is no next number of items corresponding to the presenteeism loss for calculating the presenteeism loss cost, that is, when i is larger than 7, the repetitive processing of steps S813 to S817 is ended, and the processing proceeds to step S818.

In step S818, the presenteeism loss cost of the entire company is calculated by summing up the presenteeism loss cost for each of the number of items corresponding to the presenteeism loss that has been calculated until the above step. Then, the presenteeism loss cost calculation unit 48 ends the processing.

The above embodiments may be modified as follows.

In the embodiments described above, the high stress determination result is given by whether the stress value of each employee exceeds the stress upper limit boundary value. Instead of the stress upper limit boundary value, the setting data storage unit 300 may store a criterion for evaluating the stress value in levels. At this time, in the high stress determination result, for example, five levels of determination from the A determination in which the stress is the lowest to the E determination in which the stress is the highest are performed in accordance with the criterion for evaluating the stress value in levels.

In the embodiments described above, the absenteeism loss cost calculation unit 45 obtains the number of absence days and the number of paid leave days taken due to reasons that match the target reasons for leave 308. When calculating the absenteeism loss cost, the absenteeism loss cost calculation unit 45 may obtain the number of leave-of-absence days or the hours for lateness and early leave in addition to the number of absence days and the number of paid leave days taken.

In the embodiments described above, the system for calculating the cost of labor productivity loss calculates the cost of labor productivity loss for all the employees in one company. The system for calculating the cost of labor productivity loss may calculate the cost of labor productivity loss for each department in the company. The cost of labor productivity loss may be calculated for a plurality of companies.

In the embodiments described above, the system for calculating the cost of labor productivity calculation calculates the cost of labor productivity loss for the employees in the company. The system for calculating the cost of labor productivity loss can calculate the cost of labor productivity loss for entities including non-business entities and public entities or organizations including members providing labor such as groups of self-employed people.

In the embodiments described above, the stress loss cost is calculated using the number of employees having stress values exceeding the stress upper limit boundary value, and the lifestyle disease loss cost is calculated using the number of employees having lifestyle disease determinations of “C” to “E”. The system for calculating the cost of labor productivity loss may set an employee having a poor stress check result and having a poor medical examination result as a person subject to the presenteeism loss cost calculation and calculate the presenteeism loss cost on the basis of the number of people subject to the presenteeism loss cost calculation and the presenteeism loss rate set by the company.

The determination of whether or not the result of the stress check is poor is based on whether or not the stress value of the employee exceeds the stress upper limit boundary value, and the determination of whether or not the result of the medical examination is poor is based on whether or not the employee has a lifestyle disease having received the determination from C to E. With the configuration described above, it is possible to extract the person subject to the presenteeism loss calculation on the basis of the criteria set by JAPAN SOCIETY OF NINGEN DOCK and calculate the presenteeism loss cost.

The determination of whether or not the result of the stress check is poor may be based on whether or not the stress value of the employee exceeds the national average value of stress values, and the determination of whether or not the result of the medical examination is poor may be based on whether or not the total value of the medical examination numerical values of the employee exceeds the national average value. With the configuration described above, it is possible to extract the person subject to the presenteeism loss calculation in accordance with the change in the employment environment in Japan and calculate the presenteeism loss cost.

The determination of whether or not the stress value exceeds the average value of the stress values of all the employees in the company and whether or not the result of the medical examination is poor may be based on whether or not the total value of the medical examination numerical values of the employees exceeds the average value of the total values of the medical examination numerical values of all the employees in the company. With the configuration described above, it is possible to appropriately extract the person subject to the presenteeism loss calculation in accordance with the difference in the employment environment for each industry or industry type and calculate the presenteeism loss cost.

1. A system for calculating cost of labor productivity loss, the system comprising:
an employment attendance information storage unit that stores pieces of item data on employment attendance information for employees in association with employee codes respectively assigned to the employees;
a medical examination result storage unit that stores pieces of item data on a medical examination for each of the employees in association with the corresponding employee code;
a stress check result storage unit that stores pieces of item data on a stress check for each of the employees in association with the corresponding employee code;
a setting data storage unit that stores setting data set for calculating cost of labor productivity loss;
a labor productivity loss cost calculation unit that calculates cost of labor productivity loss from the item data stored in the employment attendance information storage unit, the item data stored in the medical examination result storage unit, and the item data stored in the stress check result storage unit; and
an output unit that outputs the cost of labor productivity loss calculated by the labor productivity loss cost calculation unit to a screen or a file in a tabular format or a graphical format.

2. The system for calculating the cost of labor productivity loss according to claim 1, wherein:
the labor productivity loss cost calculation unit includes
an absenteeism loss cost calculation unit that calculates an absenteeism loss cost on a basis of the item data stored in the employment attendance information storage unit,
an overtime loss cost calculation unit that calculates an overtime loss cost on a basis of the item data stored in the employment attendance information storage unit, and
a presenteeism loss cost calculation unit that calculates a presenteeism loss cost by using the item data stored in the medical examination result storage unit and the item data stored in the stress check result storage unit as samples for specifying a presenteeism loss rate; and
the labor productivity loss cost calculation unit calculates, as the cost of labor productivity loss, a sum of the absenteeism loss cost calculated by the absenteeism loss cost calculation unit, the presenteeism loss cost calculated by the presenteeism loss cost calculation unit, and the overtime loss cost calculated by the overtime loss cost calculation unit.

3. The system for calculating the cost of labor productivity loss according to claim 1, wherein:
the labor productivity loss cost calculation unit includes
an absenteeism loss cost calculation unit that calculates an absenteeism loss cost on a basis of the item data stored in the employment attendance information storage unit,
an overtime loss cost calculation unit that calculates an overtime loss cost on a basis of the item data stored in the employment attendance information storage unit,
a lifestyle disease loss cost calculation unit that calculates a lifestyle disease loss cost on a basis of the item data stored in the medical examination result storage unit, and a stress loss cost calculation unit that calculates a stress loss cost on a basis of the item data stored in the stress check result storage unit; and
the labor productivity loss cost calculation unit calculates the cost of labor productivity loss with following Equations 1 and 2:
Presenteeism loss cost = Lifestyle disease loss cost + Stress loss cost ... (Equation 1)
Cost of labor productivity loss = Absenteeism loss cost + Presenteeism loss cost + Overtime loss cost ... (Equation 2).

4. The system for calculating the cost of labor productivity loss according to claim 3, wherein:
the stress loss cost calculation unit calculates a stress value of each of the employees on a basis of the item data stored in the stress check result storage unit, and
when an employee of which the stress value is larger than a stress upper limit boundary value included in the setting data is defined as a person with high stress, the stress loss cost calculation unit calculates the stress loss cost with Equation 3 shown below on a basis of a number of people with high stress that is the number of persons with high stress, a stress loss rate stored in the setting data storage unit, an average individual monthly unit price that is an average amount of salary paid to one employee per month, and the number of labor productivity loss cost calculation months:
Stress loss cost = Number of people with high stress × Number of calculation months × Average individual monthly unit price × Stress loss rate ... (Equation 3).

5. The system for calculating the cost of labor productivity loss according to claim 3, wherein
when an employee determined to have a lifestyle disease on a basis of the item data stored in the medical examination result storage unit and a criteria determination table for each of medical examination items is defined as a person with a lifestyle disease, the lifestyle disease loss cost calculation unit calculates the lifestyle disease loss cost with Equation 4 shown below on a basis of a number of people with a lifestyle disease that is the number of persons with a lifestyle disease, a lifestyle disease loss rate stored in the setting data storage unit, an average individual monthly unit price that is an average amount of salary paid to one employee per month, and the number of labor productivity loss cost calculation months:
Lifestyle disease loss cost = Number of people with a lifestyle disease × Number of calculation months × Average individual monthly unit price × Lifestyle disease loss rate ... (Equation 4).

6. The system for calculating the cost of labor productivity loss according to claim 2, wherein
the absenteeism loss cost calculation unit calculates the absenteeism loss cost with Equation 5 shown below on a basis of the number of paid leave days due to poor health of an employee and the number of absence days obtained from the employment attendance information storage unit, and an average individual daily unit price that is an average amount of salary paid to one employee per day, in which the average individual daily unit price is included in the setting data:
Absenteeism loss cost = (Number of absence days + Number of paid leave days due to poor health) × Average individual daily unit price ... (Equation 5).

7. The system for calculating the cost of labor productivity loss according to claim 2, wherein
the overtime loss cost calculation unit calculates the overtime loss cost with Equation 6 shown below on a basis of overtime hours including overtime work and legal overtime of an employee obtained from the employment attendance information storage unit:
Overtime loss cost = Overtime hours of all employees in calculation period × Average individual hourly unit price ... (Equation 6).

8. A method for calculating cost of labor productivity loss, the method comprising:
calculating cost of labor productivity loss with a labor productivity loss cost calculation unit by using pieces of item data on employment attendance information for employees in association with employee codes respectively assigned to the employees, pieces of item data on a medical examination for each of the employees in association with the corresponding employee code, pieces of item data on a stress check for each of the employees in association with the corresponding employee code, and setting data set for calculating cost of labor productivity loss; and
outputting the calculated cost of labor productivity loss with an output unit to a screen or a file in a tabular format or a graphical format.

9. A program for calculating cost of labor productivity loss, the program causing a computer to function as:
an employment attendance information storage unit that stores pieces of item data on employment attendance information for employees in association with employee codes respectively assigned to the employees;
a medical examination result storage unit that stores pieces of item data on a medical examination for each of the employees in association with the corresponding employee code;
a stress check result storage unit that stores pieces of item data on a stress check for each of the employees in association with the corresponding employee code;
a setting data storage unit that stores setting data set for calculating cost of labor productivity loss;
a labor productivity loss cost calculation unit that calculates cost of labor productivity loss from the item data stored in the employment attendance information storage unit, the item data stored in the medical examination result storage unit, and the item data stored in the stress check result storage unit; and
an output unit that outputs the cost of labor productivity loss output from the labor productivity loss cost calculation unit to a screen or a file in a tabular format or a graphical format.
